(19) United States
(12) Patent Application Publication
(10) Pub. No.: US 2023/0117563 A1
(43) Pub. Date: Apr. 20, 2023

(54) NEEDLE BASE UNIT, NEEDLE BASE UNIT PRODUCTION METHOD AND SYRINGE DEVICE

(71) Applicant: ASTI CORPORATION, Hamamatsu-shi, Shizuoka (JP)

(72) Inventors: Akinori INOH, Hamamatsu-shi, Shizuoka (JP); Isamu NONAKA, Hamamatsu-shi, Shizuoka (JP); Noriyuki OGAI, Hamamatsu-shi, Shizuoka (JP)

(21) Appl. No.: 17/915,203

(22) PCT Filed: Mar. 30, 2021
(86) PCT No.: PCT/JP2021/013541

§ 371 Date: Sep. 28, 2022

(30) Foreign Application Priority Data
Mar. 31, 2020 (JP) 2020-063056

(51) Int. Cl. A61M 5/32; A61M 5/34

(52) U.S. Cl. CPC A61M 5/329; A61M 5/34

(57) ABSTRACT

The purpose of the present invention is to provide a needle hub unit and the manufacturing method thereof, that enables injection with the least possible waste of a medicinal solution. The present invention is provided with a needle hub, a recess provided in the needle hub and into which a tip portion of an injection cylinder is inserted, an elastic body internally provided in the recess and fills a gap between the tip of the injection cylinder and the recess, and an injection needle inserted into the needle hub and penetrating through the elastic body, whereby the injected medicinal solution passes only through the injection needle, thus the remaining of the medicinal solution in the needle hub is prevented.

### TECHNICAL FIELD

The present invention relates to a needle hub unit, a needle hub unit manufacturing method, and an injection device, and in particular, the present invention relates to those, by which the injection can be performed with the least possible waste of medicinal solution.

### BACKGROUND ART

A conventional injection needle is disclosed, for example, in Patent Document 1.

According to an invention of “medicinal solution injection device and an injection needle used for that device” described in Patent Document 1, there is a hub to which a needle is attached. A taper-shaped recess is formed in the hub, and the tip of a syringe is inserted into the taper-shaped recess. When the hub is attached to the syringe, first, a filling material is inserted into the taper-shaped recess, and thereafter, the tip of the syringe is inserted into the taper-shaped recess. A through hole communicating with the needle is formed in the filling material.

### REFERENCE DOCUMENTS OF CONVENTIONAL ART

### Patent Document(s)

Patent Document 1: Official Gazette, Japan Patent Publication No. 2018-93906A.

### SUMMARY OF THE INVENTION

### Problems to be Solved by Invention

However, the configuration according to the conventional art as described above has the following problem.

That is, the filling material, which is to be inserted into the taper-shaped hub of the hub, includes the through hole formed therein, and consequently, there is a problem that, when the medicinal solution in the syringe is pushed out, a part of the medicinal solution remains unused in the through hole, and the medicinal solution cannot be used completely.

In the light of the above problem, it is an object of the present invention to provide a needle hub unit, a needle hub unit manufacturing method, and an injection device, by which the injection can be performed with the least possible waste of medicinal solution.

### Means to Solve the Problem

To achieve the objects mentioned above, according to claim 1 of the present invention, there is a needle hub unit, provided with: a needle hub; a recess provided in the needle hub and into which a tip portion of an injection cylinder is inserted; an elastic body internally provided in the recess and which fills a gap between the tip of the injection cylinder and the recess; and an injection needle inserted into the needle hub and penetrating through the elastic body.

Moreover, according to claim 2 of the present invention, with regard to the needle hub unit as claimed in claim 1, the base end of the injection needle is formed in a shape of a needle, and a portion formed in the shape of the needle penetrates through and protrudes from the elastic body.

Moreover, according to claim 3 of the present invention, with regard to the needle hub unit as claimed in claim 1, the base end of the injection needle is aligned with a surface of the elastic body on the side of the injection cylinder.

Moreover, according to claim 4 of the present invention, with regard to the needle hub unit as claimed in claim 3, the base end of the injection needle is cut to form a flat end.

Moreover, according to claim 5 of the present invention, with regard to the needle hub unit as claimed in claim 3, the base end of the injection needle is formed in a shape of a needle, and a space is formed in the elastic body surrounding the base end of the injection needle.

Moreover, according to claim 6 of the present invention, with regard to the needle hub unit as claimed in any one Claim among claim 1 to claim 5, an engagement portion is provided on the outer peripheral surface of the elastic body, and the engagement portion is engaged with an engagement portion provided on the side of the recess, so as to prevent the elastic body from dropping off.

Moreover, according to claim 7 of the present invention, with regard to the needle hub unit as claimed in any one Claim among claim 1 to claim 5, the elastic body is provided with a thin thickness portion, and the injection needle penetrates through the thin thickness portion.

Moreover, according to claim 8 of the present invention, with regard to the needle hub unit as claimed in any one Claim among claim 1 to claim 5, the injection needles are provided in a plural number.

Moreover, according to claim 9 of the present invention, a needle hub unit manufacturing method, for manufacturing the needle hub unit as claimed in any one Claim among claim 1 to claim 5, is provided, characterized in that: the injection needle has been provided internally in the needle hub; and the elastic body is inserted into the recess of the needle hub, whereby a base portion of the injection needle penetrates through the elastic body.

Moreover, according to claim 10 of the present invention, with regard to the needle hub unit manufacturing method as claimed in claim 9, recesses are provided in the central portion of both end surfaces, respectively disposed in the elastic body along the direction of the injection needle, whereby the base end of the injection needle penetrates through the positions of the respective recesses.

Moreover, according to claim 11 of the present invention, a needle hub unit manufacturing method, for manufacturing the needle hub unit as claimed in any one Claim among claim 1 to claim 5, is provided, characterized in that: the base portion of the injection needle and the elastic body have been formed integrally in advance, by penetration of the base portion of the injection needle through the elastic body; and the injection needle and the elastic body, formed integrally, is inserted into the recess of the needle hub.

And moreover, according to claim 12, there is an injection device provided with: the needle hub unit as claimed in any one Claim among claim 1 to claims 5; an injection cylinder inserted into the recess until the tip of the injection cylinder abuts on the elastic body; and a piston inserted into the injection cylinder until the tip of the piston abuts on the elastic body.

### Effect of the Invention

As described above, according to claim 1 of the present invention, there is a a needle hub unit, provided with: a needle hub; a recess provided in the needle hub and into which a tip portion of an injection cylinder is inserted; an elastic body internally provided in the recess and which fills a gap between the tip of the injection cylinder and the recess; and an injection needle inserted into the needle hub and penetrating through the elastic body. Therefore, during injection, the medicinal solution passes only through the inside of the injection needle, whereby the injection can be performed with the least possible waste of medicinal solution.

Moreover, according to claim 2 of the present invention, with regard to the needle hub unit as claimed in claim 1, the base end of the injection needle is formed in a shape of a needle, and a portion formed in the shape of the needle penetrates through and protrudes from the elastic body. Therefore, during assembling of the needle hub unit, the needle can be penetrated through the elastic body easily, whereby the assembling thereof can be facilitated.

Moreover, according to claim 3 of the present invention, with regard to the needle hub unit as claimed in claim 1, the base end of the injection needle is aligned with a surface of the elastic body on the side of the injection cylinder. Therefore, a piston of a syringe can be moved forward until reaching the surface of the elastic body on the side of the injection cylinder, whereby the injection can be performed with the further reduction of the waste of medicinal solution.

Moreover, according to claim 4 of the present invention, with regard to the needle hub unit as claimed in claim 3, the base end of the injection needle is cut to form a flat end. Therefore, the effect of the present invention as described above can be secured further.

Moreover, according to claim 5 of the present invention, with regard to the needle hub unit as claimed in claim 3, the base end of the injection needle is formed in a shape of a needle, and a space is formed in the elastic body surrounding the base end of the injection needle. Therefore, the effect of the present invention as described above can be secured further.

Moreover, according to claim 6 of the present invention, with regard to the needle hub unit as claimed in any one Claim among claim 1 to claim 5, an engagement portion is provided on the outer peripheral surface of the elastic body, and the engagement portion is engaged with an engagement portion provided on the side of the recess, so as to prevent the elastic body from dropping off. Therefore, the elastic body can be held securely.

Moreover, according to claim 7 of the present invention, with regard to the needle hub unit as claimed in any one Claim among claim 1 to claim 5, the elastic body is provided with a thin thickness portion, and the injection needle penetrates through the thin thickness portion. Therefore, the injection needle can be penetrated through the elastic body easily, whereby the assembling thereof can be facilitated.

Moreover, according to claim 8 of the present invention, with regard to the needle hub unit as claimed in any one Claim among claim 1 to claim 5, the injection needles are provided in a plural number. Therefore, the injection can be performed effectively.

Moreover, according to claim 9 of the present invention, a needle hub unit manufacturing method, for manufacturing the needle hub unit as claimed in any one Claim among claim 1 to claim 5, is provided, characterized in that: the injection needle has been provided internally in the needle hub; and the elastic body is inserted into the recess of the needle hub, whereby a base portion of the injection needle penetrates through the elastic body. Therefore, the assembling thereof can be facilitated.

Moreover, according to claim 10 of the present invention, with regard to the needle hub unit manufacturing method as claimed in claim 9, recesses are provided in the central portion of both end surfaces, respectively disposed in the elastic body along the direction of the injection needle, whereby the base end of the injection needle penetrates through the positions of the respective recesses. Therefore, the injection needle can be penetrated straight, through the center of the elastic body.

Moreover, according to claim 11 of the present invention, a needle hub unit manufacturing method, for manufacturing the needle hub unit as claimed in any one Claim among claim 1 to claim 5, is provided, characterized in that: the base portion of the injection needle and the elastic body have been formed integrally in advance, by penetration of the base portion of the injection needle through the elastic body; and the injection needle and the elastic body, formed integrally, is inserted into the recess of the needle hub. Therefore, the assembling thereof can be facilitated.

And moreover, according to claim 12, there is an injection device provided with: the needle hub unit as claimed in any one Claim among claim 1 to claims 5; an injection cylinder inserted into the recess until the tip of the injection cylinder abuts on the elastic body; and a piston inserted into the injection cylinder until the tip of the piston abuts on the elastic body. Therefore, the waste of medicinal solution can be eliminated.

### BRIEF DESCRIPTION OF DRAWINGS

FIG. 1 is a longitudinal sectional view according to a first embodiment of the present invention, showing a needle hub unit into which a tip of an injection cylinder is inserted.

FIG. 2 are views according to the first embodiment of the present invention, showing procedure for of assembling of the needle hub unit, in which: FIG. 2 (a) is a longitudinal sectional view showing a state that an elastic body is about to be inserted into the needle hub; and FIG. 2 (b) is a longitudinal sectional view showing a state that the elastic body has been inserted into the needle hub.

FIG. 3 is a longitudinal sectional view according to a second embodiment of the present invention, showing a needle hub unit into which a tip of an injection cylinder is inserted.

FIG. 4 are views according to the second embodiment of the present invention, showing procedure for assembling of the needle hub unit, in which: FIG. 4 (a) is a longitudinal sectional view showing a state that an elastic body is about to be inserted into the needle hub; and FIG. 4 (b) is a longitudinal sectional view showing a state that the elastic body has been inserted into the needle hub.

FIG. 5 is a longitudinal sectional view of a needle hub unit according to a third embodiment of the present invention.

FIG. 6 is a longitudinal sectional view according to a fourth embodiment of the present invention, showing a needle hub unit into which a tip of an injection cylinder is inserted.

FIG. 7 is a longitudinal sectional view of a needle hub unit according to a fifth embodiment of the present invention.

FIG. 8 are views according to a sixth embodiment of the present invention, in which: FIG. 8 (a) is a sectional view showing a state that, an elastic body is inserted into a needle hub, and thereafter an injection cylinder is inserted therein; FIG. 8 (b) is a sectional view showing a state that. the elastic body is inserted into the needle hub, and thereafter the injection cylinder is inserted therein, and thereafter, a piston is inserted therein; and FIG. 8 (c) is a sectional view showing a state that, the elastic body is inserted into the needle hub, and thereafter the injection cylinder is inserted therein, and thereafter, the piston is inserted therein until the piston abuts on the elastic body.

FIG. 9 is a sectional view according to a seventh embodiment of the present invention, showing a state that, an elastic body is inserted into a needle hub, and thereafter an injection cylinder is inserted therein, and thereafter, a piston is inserted therein until the piston abuts on the elastic body.

FIG. 10 are views according to an eighth embodiment of the present invention, in which: FIG. 10 (a) is a longitudinal sectional view showing a state that an elastic body is about to be inserted into a needle hub; and FIG. 10 (b) is a longitudinal sectional view showing a state that the elastic body has been inserted into the needle hub.

### MODE(S) FOR CARRYING OUT THE INVENTION

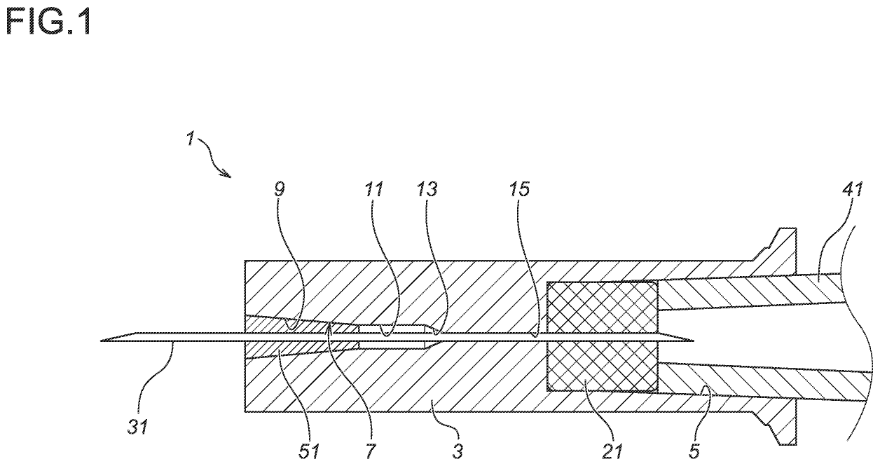

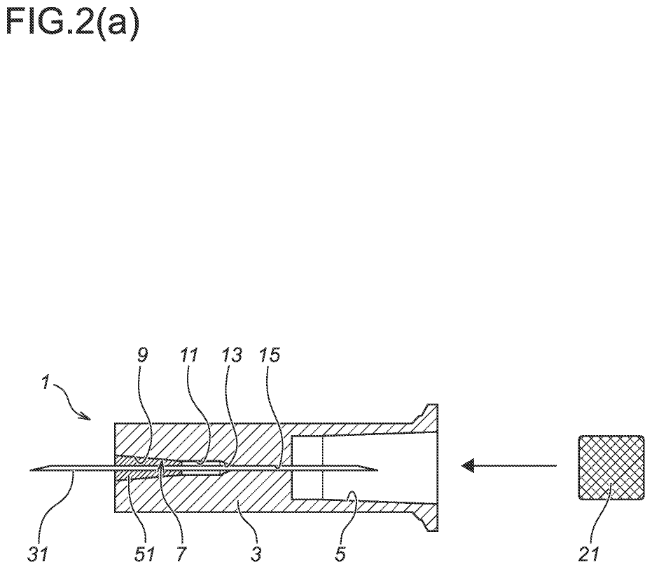

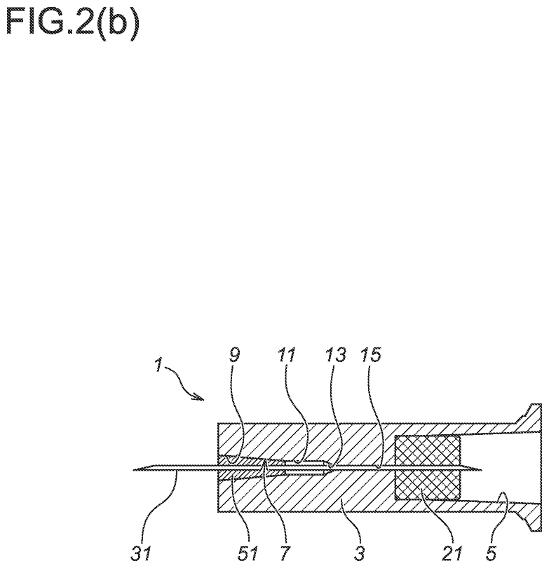

Now, a first embodiment of the present invention will be explained with reference to FIG. 1 and FIG. 2.

First, as illustrated in FIG. 1, a needle hub unit 1 includes a needle hub 3. An injection cylinder insertion recess 5 is formed on the right side of the needle hub 3 as shown in FIG. 1. The injection cylinder insertion recess 5 is in a taper shape, of which diameter increases gradually toward the side of an opening (on the right side of FIG. 1).

Moreover, a needle insertion through hole 7 is formed on the left side of the needle hub 3. The needle insertion through hole 7 and the injection cylinder insertion recess 5 are communicating with each other. The left side of the needle insertion through hole 7 as shown in FIG. 1 constitutes a taper-shaped portion 9, of which diameter increases gradually toward the side of an opening (on the left side of FIG. 1). A first straight portion 11 is formed, of which diameter remains unchanged at the same diameter as that of the tape-shaped portion 9 on the side opposite to the opening (at the right end of FIG. 1), in the vicinity of the center of the needle insertion through hole 7 in the right and left directions of FIG. 1. The right side of the first straight portion 11 of FIG. 1 constitutes a diameter decreasing portion 13, of which diameter decreases gradually toward the right side of FIG. 1. A second straight portion 15 as an open hole, of which diameter remains unchanged at the same diameter as that of the diameter decreasing portion 13 on the side opposite to the opening (at the right end of FIG. 1), is formed in the needle insertion through hole 7, on the right side of the diameter decreasing portion 13 as shown in FIG. 1.

Moreover, the needle unit 1 includes an elastic body 21. The elastic body 21 is made of, for example, silicone rubber, butyl rubber or thermoplastic elastomer, and is inserted until reaching the deepest end (at the left end of FIG. 1) of the injection cylinder insertion recess 5 of the needle hub 3.

Moreover, the needle hub unit 1 includes an injection needle 31. The injection needle 31 is inserted into the needle insertion through hole 7 of the needle hub 3, and is installed in the needle hub 3. Moreover, the base end side (on the right side of FIG. 1) of the injection needle 31 penetrates through the elastic body 21, and protrudes into the inside of the injection cylinder insertion recess 5. Moreover, the both ends of the injection needle 31 are formed in the shape of needles, respectively.

The needle hub unit 1 is attached to the injection cylinder 41, with the press fitting of the tip of the injection cylinder 41 into the injection cylinder insertion recess 5. As illustrated in FIG. 1, the injection cylinder 41 is press-fitted into the injection cylinder insertion recess 5, until the top of the injection cylinder 41 depresses the elastic body 21. Moreover, when the needle hub unit 1 is attached to the injection cylinder 41, the base end side of the injection needle 31 (on the right side of FIG. 1) protrudes into the inside of the injection cylinder 41.

An adhesive 51 is filled into a gap between the injection needle 31 and the inner peripheral surface of the needle insertion through hole 7, and by means of the adhesive 51, the injection needle 31 is held in the injection cylinder 41.

Next, the function of the first embodiment will be explained.

The assembling of the needle hub unit 1 is performed as shown in FIG. 2.

First, as illustrated in FIG. 2 (a), the injection needle 31 is inserted into the needle insertion through hole 7 of the needle hub 3, and the adhesive 51 is filled in the gap between the injection needle 31 and the inner peripheral surface of the needle insertion through hole 7.

Next, as illustrated in FIG. 2 (b), the elastic body 21 is inserted into the injection cylinder insertion (press-fitting) recess 5 of the needle hub 3. In such a situation, the injection needle 31 penetrates through the elastic body 21, and the base end side of the injection needle 31 (on the right side of FIG. 2 (b)) protrudes into the inside of the injection cylinder insertion recess 5.

When the needle hub unit 1 is used, as illustrated in FIG. 1, the injection cylinder 41 is press-fitted into the injection cylinder insertion recess 5, until the tip of the injection cylinder 41 depresses the elastic body 21.

Next, the effect of the first embodiment will be explained.

First, there is the elastic body 21 in the injection cylinder insertion recess 5 of the needle hub 3, the injection needle 31 penetrates through the elastic body 21, and when the needle hub unit 1 is attached to the injection cylinder 41, the base end side of the injection needle 31 (on the right side of FIG. 1) protrudes into the inside of the injection cylinder 41. Consequently, a medicinal solution in the injection cylinder 41 is injected by passing only through the inside of the injection needle 31, and no medicinal solution is left unused in the needle hub unit 1, and therefore, the injection can be performed with the least possible waste of the medicinal solution.

Moreover, since the protruding portion on the base end side of the injection needle 31 is formed in the shape of needle, there is no medicinal solution left in that portion.

And moreover, since the both ends of the injection needle 31 are in the form of needles, respectively, when assembling of the needle hub unit 1, the injection needle 31 can penetrate through the elastic body 21 easily, whereby the assembling thereof can be facilitated.

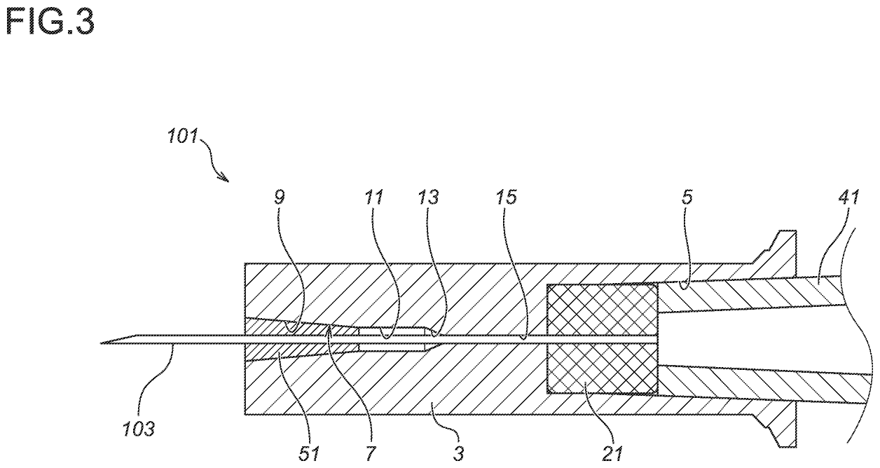

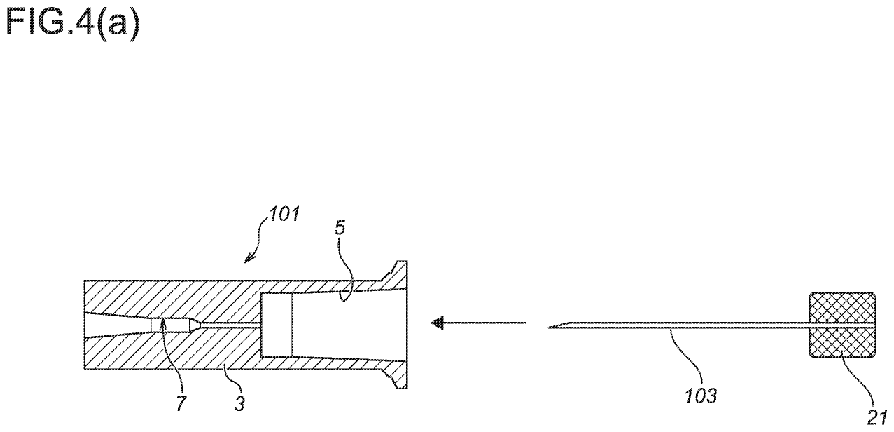

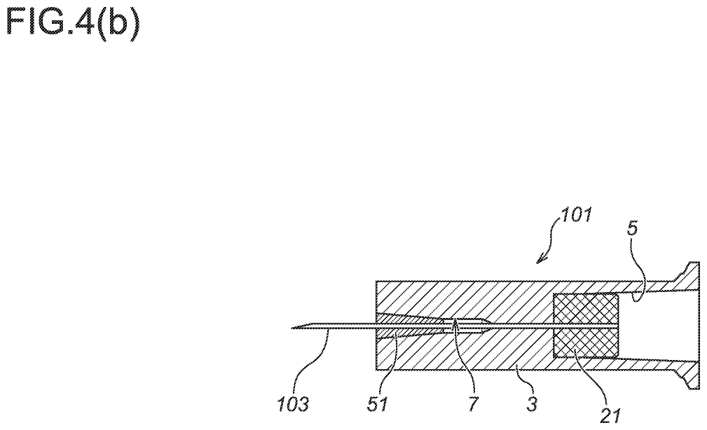

Next, a second embodiment of the present invention will be explained with reference to FIG. 3 and FIG. 4.

The structure of a needle hub unit 101 according to the second embodiment is substantially the same as that of the needle hub unit 1 of the first embodiment. However, there is a different feature that, an injection needle 103 having a different structure is installed in the needle hub 3. With regard to the injection needle 103, only the tip side (on the left side of FIG. 3) is formed in the shape of needle, and the base end side (on the right side of FIG. 3) is cut to form a flat end. Further, with regard to the injection needle 103, the base end side (on the right side of FIG. 3) is aligned with the end surface of the elastic body 21 on the side of the injection cylinder 41 (on the right side of FIG. 3), and does not protrude into the side of the injection cylinder 41.

Next, the function of the second embodiment will be explained.

The assembling of the needle hub unit 101 is performed as shown in FIG. 4.

First, as illustrated in FIG. 4 (a), the tip side of the injection needle 31 (on the left side of FIG. 4 (a)) is pierced from the right side of the elastic body 21 as shown in FIG. 4 (a), and is moved until the end surface of the injection needle 31 on the base end surface (on the right side of FIG. 4 (a)) becomes aligned with the end surface of the elastic body 21 on the right side of FIG. 4 (a), to form a uniform surface with each other.

Next, as illustrated in FIG. 4 (b), the tip side of the injection needle 31 (on the left side of FIG. 4 (b)) is inserted into the needle insertion through hole 7, from the side of the injection cylinder insertion recess 5 of the needle hub 3 (on the right side of FIG. 4 (b)), and the elastic body 21 is inserted into the deepest end of the injection cylinder insertion recess 5 (on the left side of FIG. 4 (b)). Thereafter, the adhesive 51 is filled in the gap between the injection needle 31 and the inner peripheral surface of the needle insertion through hole 7.

Furthermore, in the case of the second embodiment, in addition to the same function and effect as those of the first embodiment, the following function and effect can be exhibited. That is, the base end side of the injection needle 103 (on the right side of FIG. 3) is aligned with the end surface of the elastic body 21 on the side of the injection cylinder 41 (on the right side of FIG. 3), and the injection needle does not protrude into the side of the injection cylinder 41. Consequently, a piston (not shown) of the injection cylinder 41 can move forward until reaching the end surface of the elastic body 21 on the side of the injection cylinder 41 (on the right side of FIG. 3), whereby the medicinal solution can be injected with the further reduction of the waste of medicinal solution. The piston is normally composed of flexible materials, and therefore, even in the case of the structure according to the first embodiment, the piston may move forward until reaching the end surface of the elastic body 21 on the side of the injection cylinder 41 (on the right side of FIG. 3). However, where the piston is composed of rigid materials, the injection needle 41 protruding into the inside of the injection cylinder 41 is unable to pierce into the piston, and the further depressing of the piston is prohibited. The present embodiment is effective in such a situation.

It should be noted that, in the drawings, the same reference signs will be allotted to the elements common to the first embodiment and the second embodiment, and the explain thereof will be omitted.

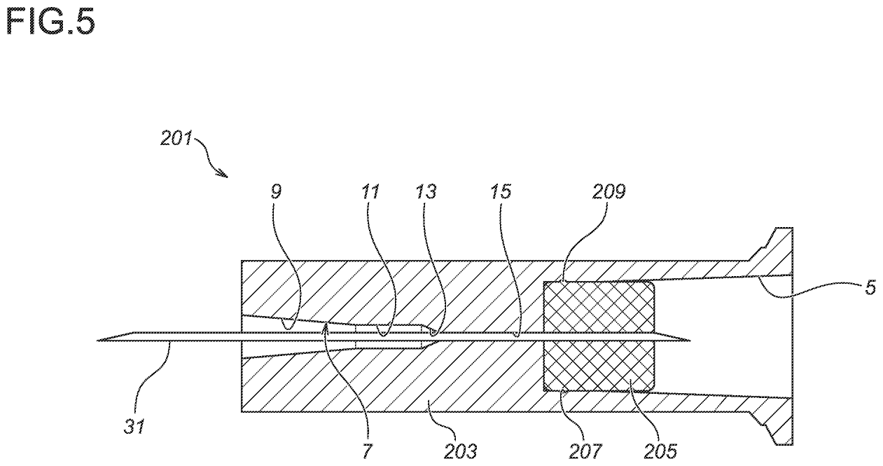

Next, a third embodiment of the present invention will be explained with reference to FIG. 5.

The structure of a needle hub unit 201 according to the third embodiment is substantially the same as that of the needle hub unit 1 of the first embodiment. However, there is a different feature that, a needle hub 203 and an elastic body 205, respectively having different structures, are used. The structure of the needle hub 203 is substantially the same as that of the needle hub 3 of the first embodiment, but an elastic engagement recess 207 in an annular shape is formed on the inner peripheral surface of the bottom side of the injection cylinder insertion recess 5 (on the left side of FIG. 5), and an elastic body engagement projection 209 is formed on the side circumferential surface of the elastic body 205. Furthermore, when the elastic body 205 is installed in the injection cylinder insertion recess 5, the elastic body engagement recess 207 and the elastic body engagement projection 209 are engaged with each other.

Next, according to the third embodiment, in addition to the effect and function of the first embodiment, the following function and effect can be further exhibited. That is, when the elastic body 205 is installed in the injection cylinder insertion recess 5, the elastic body engagement recess 207 and the elastic body engagement projection 209 are engaged with each other, and consequently, the elastic body 205 can be held securely.

It should be noted that, in the drawings, the same reference signs will be allotted to the elements common to the first embodiment and the third embodiment, and the explain thereof will be omitted.

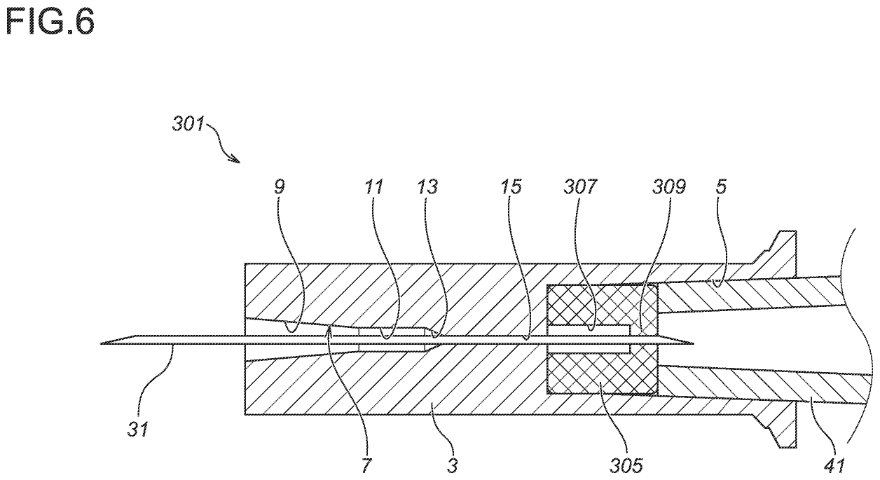

Next, a fourth embodiment of the present invention will be explained with reference to FIG. 6.

The structure of a needle hub unit 301 according to the fourth embodiment is substantially the same as that of the needle hub unit 1 of the first embodiment. However, there is a different feature that, an elastic body 305 having a different structure is used. With regard to the elastic body 305, a recess 307, having an opening toward the bottom side of the injection cylinder insertion recess 5 (on the left side of FIG. 6), is formed, and a thin thickness portion 309 is formed on the bottom side of the recess 307 (on the right side of FIG. 6). The base end side of the injection needle 31 (on the right side of FIG. 6) penetrates through the thin thickness portion 309, and is exposed on the side of the injection cylinder insertion recess 5.

Next, according to the fourth embodiment, in addition to the effect and function of the first embodiment, the following function and effect can be further exhibited. That is, the base end side of the injection needle 31 (on the right side of FIG. 6) penetrates through the thin thickness portion 309 of the elastic body 305, and is exposed on the side of the injection cylinder insertion recess 5, and consequently, the injection needle 31 can penetrate through the elastic body 305 easily. Therefore, when the needle hub unit 301 is assembled, the installation of the elastic body 305 in the injection cylinder insertion recess 5 can be performed easily, and consequently, the assembling of the needle hub unit 301 can be facilitated.

It should be noted that, in the drawings, the same reference signs will be allotted to the elements common to the first embodiment and the fourth embodiment, and the explain thereof will be omitted.

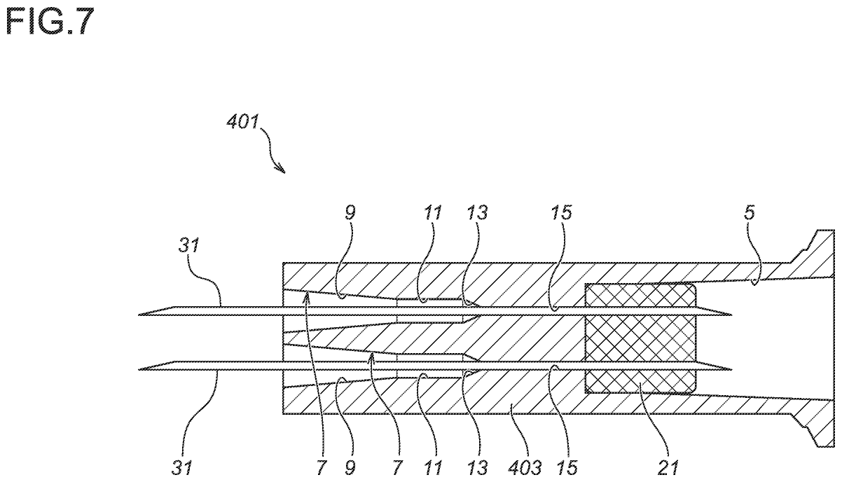

Next, a fifth embodiment of the present invention will be explained with reference to FIG. 7.

The structure of a needle hub unit 401 according to the fifth embodiment is substantially the same as that of the needle hub unit 1 of the first embodiment. However, there is a different feature that, a needle hub 403 having a different structure is used. The structure of the needle hub 403 is substantially the same as that of the needle hub 3 of the first embodiment, but a plurality of (in the fifth embodiment, two) needle insertion through holes 7 are formed, and a plurality of (in the fifth embodiment, two) injection needles 31 are installed.

Next, according to the fifth embodiment, in addition to the effect and function of to the first embodiment, the following function and effect can be further exhibited. That is, since a plurality of (in the fifth embodiment, two) injection needles 31 are installed, the injection can be performed more effectively.

It should be noted that, in the drawings, the same reference signs will be allotted to the elements common to the first embodiment and the fifth embodiment, and the explain thereof will be omitted.

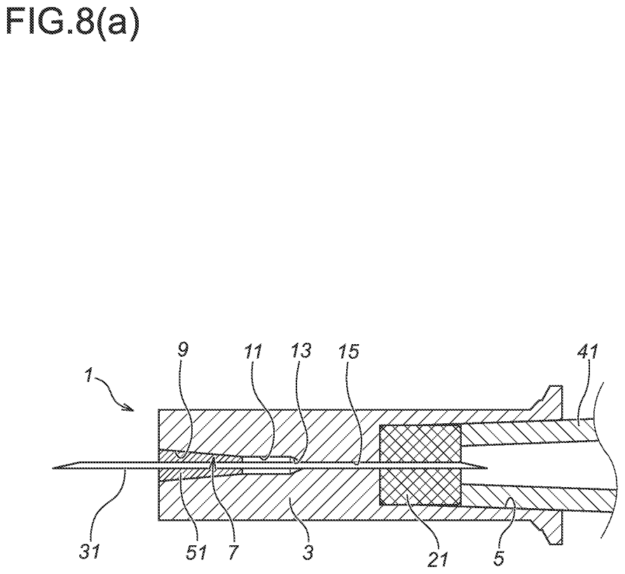

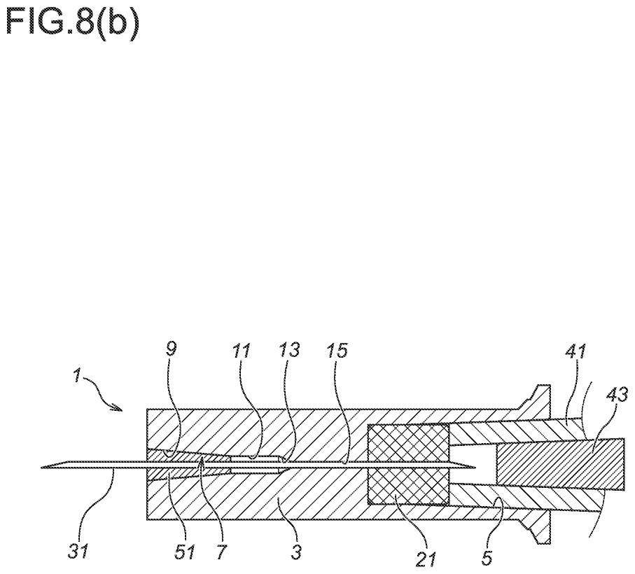

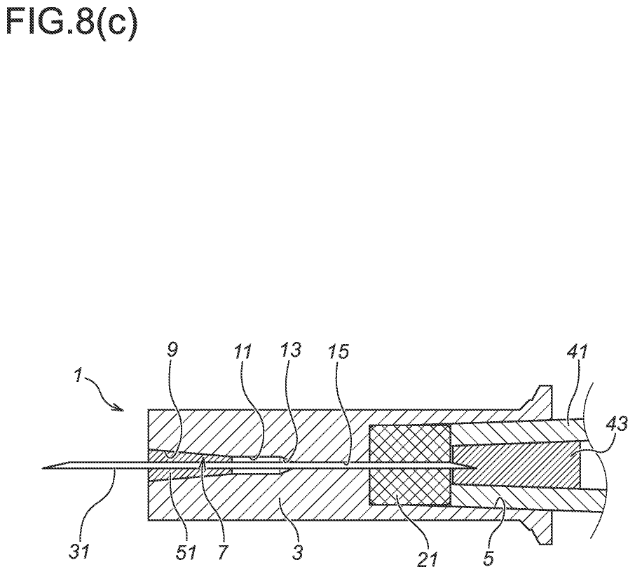

Next, a sixth embodiment of the present invention will be explained with reference to FIG. 8. In the case of the first embodiment, there is no specific requirement for the structure of the piston, which is inserted into the injection cylinder. However, in the present embodiment, as illustrated in FIG. 8 (c), there is a prerequisite that a piston 43, which is inserted into the injection cylinder 41, can be inserted until the tip of the piston 43 abuts on the elastic body 21.

For reference, the other structure is substantially the same as that of the first embodiment.

According to the above structure, when the injection needle 31 is put into a medicinal solution bottle (not shown) and the piston 43 is pulled, the medicinal solution in the medicinal solution bottle is sucked into the injection cylinder 41.

For reference, if the injection needle 31 is thin, the medicinal solution is sucked into the injection cylinder 41 by using another needle hub 3 provided with a thick injection needle 31, and thereafter, the replacement to the needle hub 3 provided with the thick injection needle 31 is performed.

Next, the injection needle 31 is punctured into a predetermined position of a human (not shown), and as illustrated in FIG. 8 (b), the piston 43 is depressed. Thereafter, as illustrated in FIG. 8 (c), the piston 43 is depressed until the tip surface of the piston 43 abuts on the surface of the elastic body 21. As a result, all the medicinal solution in the injection cylinder 41 can be ejected completely.

Therefore, substantially the same effect as that of the first embodiment can be accomplished, and in addition, since the piston 43 can be depressed until abutting on the elastic body 21, the waste of medicinal solution can be reduced further.

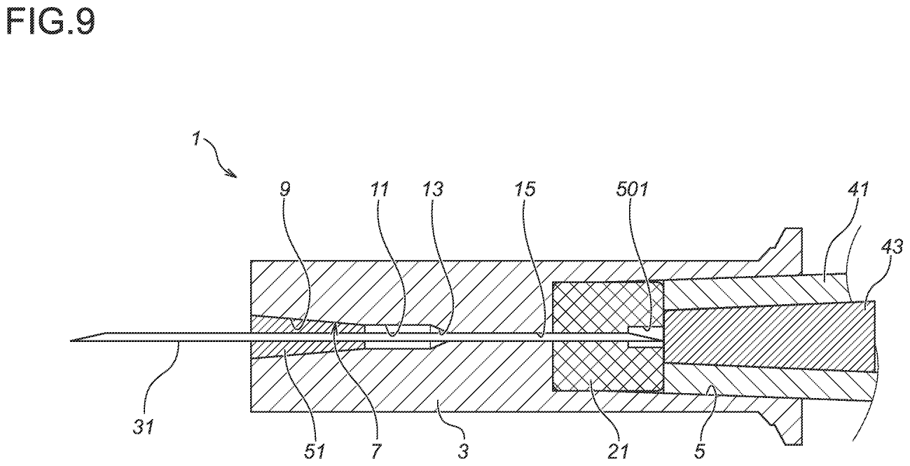

Next, a seventh embodiment of the present invention will be explained with reference to FIG. 9. In the present embodiment, substantially the same as the case of the second embodiment, the base end side of the injection needle 103 (on the right side of FIG. 9) is aligned with the end surface of the elastic body 21 on the side of the injection cylinder 41 (on the right side of FIG. 9), and does not protrude into the side of the injection cylinder 41. However, and moreover, the base end of the injection needle 31 is not in a flat shape, but has been cut off obliquely. Moreover, a recess 501 is formed on the end surface of the elastic body 21, surrounding the base end portion of the injection needle 31.

The other structure is substantially the same as that of the second embodiment as described above, thus the same reference signs will be allotted to the common elements in the drawing, and the explain thereof will be omitted.

Since the base end side of the injection needle 103 (on the right side of FIG. 9) is aligned with the end surface of the elastic body 21 on the side of the injection cylinder 41 (on the right side of FIG. 9), the piston 43 can be depressed until abutting on the elastic body 21. Moreover, the base end side of the injection needle 31 is not in a flat shape, but has been cut off obliquely, and consequently, for the purpose of manufacturing, the same method as that of the first embodiment can be adopted. Moreover, since the recess 501 is formed, the medicinal solution can be introduced into the injection needle 31 securely.

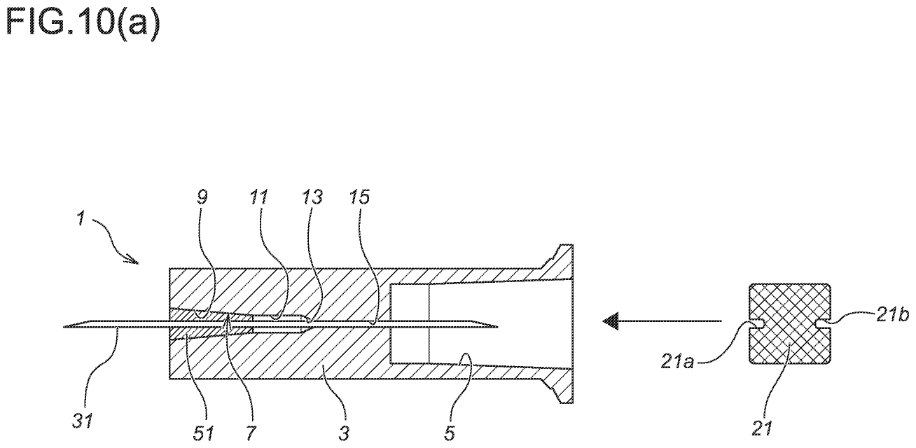

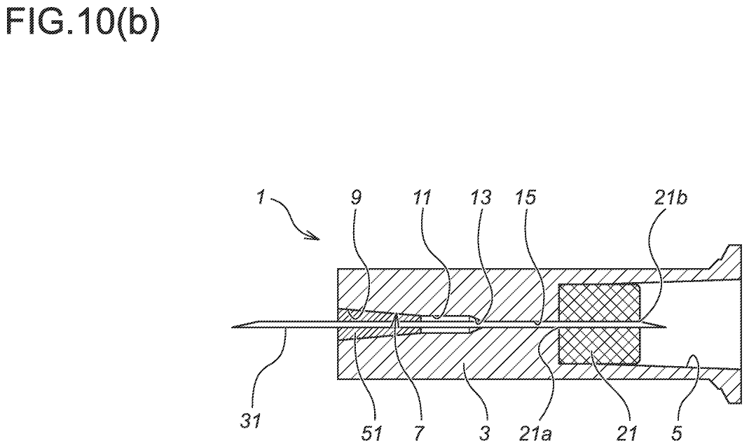

Next, an eighth embodiment of the present invention will be explained with reference to FIG. 10. With regard to the structure of the first embodiment as described above, the present embodiment has a structure that recesses 21a, 21b are provided at the center position of the both end surfaces along the insertion direction of the elastic body 21.

For reference, the other structure is substantially the same as that of the first embodiment as described above, thus the same reference signs will be allotted to the common elements in the drawing, and the explain thereof will be omitted.

According to the structure as described above, since the recesses 21a, 21b are provided at the center position of the both end surfaces along the insertion direction of the elastic body 21, there is a merit that the base end portion of the injection needle 31 is guided to the center of the elastic body 21, and straight penetration thereof at the correct angle can be performed. In addition, substantially the same effect as that of the first embodiment can be accomplished.

For reference, each of the recesses 21a, 21b is substantially in the same size as, or slightly larger than, the outer diameter of the injection needle 31. Therefore, when the elastic body 21 is deformed by the injection needle, the recesses 21a, 21b becomes filled up.

The present invention is not limited to the first to the eighth embodiments as described above.

Any other rubber, elastomer, etc., may be used as an elastic member.

The needle hub and the injection needle may be produced integrally by means of insert molding.

Various materials, sizes, and numbers may be adopted for each of the structural elements.

Furthermore, the structures shown in the drawings are merely for example purposes only.

### INDUSTRIAL APPLICABILITY

The present invention relates to the needle hub unit, the needle hub unit manufacturing method, and the injection device, and in particular, the present invention relates to those, by which the injection can be performed with the least possible waste of medicinal solution. The present invention is suitable, for example, for a syringe for the injection of Botox (trademark, botulinum toxin type A).

### EXPLANATION OF REFERENCE NUMERALS AND SIGNS

1 Needle Hub Unit

5 Injection Cylinder Insertion Recess

21 Elastic Body

41 Injection Cylinder

31 Injection Needle

101 Needle Hub Unit

103 Injection Needle

201 Needle Hub Unit

207 Elastic Body Engagement Recess

209 Elastic Body Engagement Projection

301 Needle hub Unit

305 Elastic Body

309 Thin Thickness Portion

401 Needle Hub Unit

1. A needle hub unit provided with:
a needle hub;
a recess provided in the needle hub and into which a tip portion of an injection cylinder is inserted;
an elastic body internally provided in the recess and which fills a gap between the tip of the injection cylinder and the recess; and
an injection needle inserted into the needle hub and penetrating through the elastic body.

2. The needle hub unit as claimed in claim 1, wherein,
the base end of the injection needle is formed in a shape of a needle, and a portion formed in the shape of the needle penetrates through and protrudes from the elastic body.

3. The needle hub unit as claimed in claim 1, wherein,
the base end of the injection needle is aligned with a surface of the elastic body on the side of the injection cylinder.

4. The needle hub unit as claimed in claim 3, wherein,
the base end of the injection needle is cut to form a flat end.

5. The needle hub unit as claimed in claim 3, wherein,
the base end of the injection needle is formed in a shape of a needle, and a space is formed in the elastic body surrounding the base end of the injection needle.

6. The needle hub unit according to claim 1, wherein,
an engagement portion is provided on the outer peripheral surface of the elastic body, and the engagement portion is engaged with an engagement portion provided on the side of the recess, so as to prevent the elastic body from dropping off.

7. The needle hub unit according to claim 1, wherein,
the elastic body is provided with a thin thickness portion, and the injection needle penetrates through the thin thickness portion.

8. The needle hub unit according to claim 1, wherein,
the injection needles are provided in a plural number.

9. A needle hub unit manufacturing method, for manufacturing the needle hub unit according to claim 1, wherein,
the injection needle has been provided internally in the needle hub; and
the elastic body is inserted into the recess of the needle hub, whereby a base portion of the injection needle penetrates through the elastic body.

10. The needle hub unit manufacturing method as claimed in claim 9, wherein,
recesses are provided in the central portion of both end surfaces, respectively disposed in the elastic body along the direction of the injection needle, whereby the base end of the injection needle penetrates through the positions of the respective recesses.

11. A needle hub unit manufacturing method, for manufacturing the needle hub according to claim 1, wherein,
the base portion of the injection needle and the elastic body have been formed integrally in advance, by penetration of the base portion of the injection needle through the elastic body; and
the injection needle and the elastic body, formed integrally, is inserted into the recess of the needle hub.

12. An injection device provided with:
the needle hub unit according to claim 1;
an injection cylinder inserted into the recess until the tip of the injection cylinder abuts on the elastic body; and
a piston inserted into the injection cylinder until the tip of the piston abuts on the elastic body.
